(19) United States
(12) Patent Application Publication
(10) Pub. No.: US 2018/0011088 A1
(43) Pub. Date: Jan. 11, 2018

(54) Methods for Immuno Chromatographic Assay Desensitization

(71) Applicant: Neogen Corporation, Lansing, MI (US)

(72) Inventors: Andrew John Wheeler, Hollis Center, ME (US); Andrew John Denhartigh, Lansing, MI (US); Frank Eric Klein, Howell, MI (US); David Phillip Ankrapp, Grand Blanc, MI (US); Danielle Alexander, Haslett, MI (US)

(21) Appl. No.: 15/546,751

(22) PCT Filed: Jan. 27, 2016
(86) PCT No.: PCT/US16/15025

§ 371 Date: Jul. 27, 2017

Related U.S. Application Data
(60) Provisional application No. 62/109,252, filed on Jan. 29, 2015.

(51) Int. Cl. G01N 33/543; G01N 33/545; G01N 33/553; B01D 15/38; G01N 33/94; G01N 33/558

(52) U.S. Cl. CPC G01N 33/54306; G01N 33/9446; G01N 33/558; G01N 33/553; B01D 15/3809; G01N 33/54386; G01N 33/54346; G01N 33/545

(57) ABSTRACT

The present disclosure provides a device and method for measuring an amount of an analyte in a sample, comprising a lateral flow matrix which defines a flow path and which comprises, in series: a sample receiving zone; a labeling zone comprising an unlabeled receptor and a labeled receptor, the unlabeled receptor located downstream of the labeled receptor and separated by a distance; and two serially oriented capture zones capable of providing quantitation of the amount of the analyte in the sample.

### TECHNICAL FIELD

This invention relates to an analyte test device and method employing a lateral-flow test strip for the detection of an analyte. More specifically, this invention relates to devices and methods for desensitizing a lateral-flow test strip.

### BACKGROUND

Lateral-flow or immunochromatographic assays and methods for the detection of the presence or concentration of analytes from liquid samples have been developed. For example, assays and methods have been used in pregnancy test kits, for the detection of antibiotics, and to detect drugs in urine and blood samples.

Government agencies throughout the world have established limits for particular residues in foodstuffs. Residues that are above a certain predetermined threshold are considered unsafe for human consumption. Many currently available assays are overly sensitive for certain residues which results in false positive test results. Thus, there is a need for a detection method that is end-user friendly and that only gives a positive result when an analyte is found to be at or above a certain concentration level in a sample.

These needs and other needs are satisfied by the devices, methods, and kits of the present invention.

### SUMMARY OF THE INVENTION

The present invention provides a lateral flow assay device, method, and kit for analyte detection. The lateral flow assay format may reduce and optimize the detection sensitivity for analytes to a desired level or threshold. The antibody used to detect the antigen is left in free form and is not directly conjugated to a substrate (i.e., the unlabeled receptor). A detector molecule may then capture that specific antibody, using, for instance, an anti-species or a tag, and may be used to measure the amount of free antibody that is bound to the immobilized antigen-protein test line.

Surprisingly, the placement of the unlabeled receptor (within the sample application zone) affects the sensitivity of the immunochromatographic test. Generally, the closer the unlabeled receptor is positioned to the labeled receptor, the more sensitive the test. The further away the unlabeled receptor is positioned to the labeled receptor, the less sensitive the test.

In one aspect, the present invention provides a device for measuring an amount of an analyte in a sample, comprising a lateral flow matrix which defines a flow path and which comprises, in series: a sample receiving zone; a labeling zone comprising an unlabeled receptor and a labeled receptor, the unlabeled receptor located downstream of the labeled receptor and separated by a distance; and two serially oriented capture zones capable of providing quantitation of the amount of the analyte in the sample.

In another aspect, a method for measuring an amount of an analyte in a sample is provided. The method includes providing a lateral flow matrix device comprising an unlabeled receptor, the unlabeled receptor located downstream of the labeled receptor and separated by a distance; contacting the sample to the lateral flow matrix device, wherein the analyte binds to at least one of the unlabeled receptor or the labeled receptor to form one or more analyte-receptor complexes; allowing the sample to come into contact with a receptor binder on a solid support, wherein the receptor binder binds to the at least one of the unlabeled or the labeled receptors but does not bind to the one or more analyte-receptor complexes; and detecting a quantity of the receptor binder bound to the at least one of the unlabeled or labeled receptors as an inverse indication of the amount of the analyte in the sample at or above a predetermined threshold level.

In still another aspect, a kit for detecting the presence of a predetermined threshold amount of an analyte in a sample is provided. The kit includes a container that comprises a lateral flow matrix which defines a flow path and which comprises in series: a sample receiving zone; a labeling zone comprising an unlabeled receptor and a labeled receptor, the unlabeled receptor located downstream of the labeled receptor and separated by a distance; and two serially oriented capture zones capable of providing quantification of the amount of analyte in the sample, wherein a result that the analyte is present in the sample at or above the predetermined threshold amount is a positive result.

Additional aspects will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

These and other features, aspects, and advantages of the present invention may be better understood when the following detailed description is read with reference to the accompanying drawings.

FIG. 1 is a top plan view of a single lane lateral flow assay device for visually quantifying analytes in accordance with one or more embodiments of the present disclosure.

FIG. 2 is a side-on view of the single lane lateral flow assay device of FIG. 1 in accordance with one or more embodiments of the present disclosure.

FIG. 3 is a series of assay results in accordance with one or more embodiments of the present disclosure.

FIG. 4 is a series of assay results in accordance with one or more embodiments of the present disclosure.

FIG. 5 is a series of assay results in accordance with one or more embodiments of the present disclosure.

FIG. 6 is a series of assay results in accordance with one or more embodiments of the present disclosure.

FIG. 7 is a graph comparing the tetracycline ratio to the concentration of tetracycline in the sample (ppb) in accordance with one or more embodiments of the present disclosure.

FIG. 8 is a graph comparing the peak height to the results obtained from cow milk (left side) and goat milk (right side) in accordance with one or more embodiments of the present disclosure.

FIG. 9 is a graph comparing the tetracycline ratio to the concentration of tetracycline in each sample (ppb), where four different spacings between the labeled and unlabeled receptors were used for each concentration of tetracycline.

FIG. 10 is a graph comparing the peak height to the concentration of chlortetracycline in the sample (ppb) in accordance with one or more embodiments of the present disclosure.

FIG. 11 is a graph comparing the peak height to the concentration of chlortetracycline in each sample (ppb), where three different spacings between the labeled and the unlabeled receptors were used for each concentration of chlortetracycline.

The figures are provided by way of example and are not intended to limit the scope of the invention.

### DETAILED DESCRIPTION

In the following description, numerous specific details are given to provide a thorough understanding of the embodiments. The embodiments can be practiced without one or more of the specific details, or with other methods, components, materials, etc. In other instances, well-known structures, materials, or operations are not shown or described in detail to avoid obscuring aspects of the embodiments.

Reference throughout this specification to “one embodiment,” “an embodiment,” or “embodiments” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless indicated otherwise, when a range of any type is disclosed or claimed, it is intended to disclose or claim individually each possible number that such a range could reasonably encompass, including any sub-ranges encompassed therein. Moreover, when a range of values is disclosed or claimed, which Applicants intend to reflect individually each possible number that such a range could reasonably encompass, Applicants also intend for the disclosure of a range to reflect, and be interchangeable with, disclosing any and all sub-ranges and combinations of sub-ranges encompassed therein. Accordingly, Applicants reserve the right to provisio out or exclude any individual numbers or ranges, including any sub-ranges or combinations of sub-ranges within the group, if for any reason the Applicants choose to claim less than the full measure of the disclosure, for example, to account for a reference that Applicants are unaware of at the time of the filing of the application.

The Abstract of this disclosure is provided for the purpose of satisfying the requirements of 37 C.F.R. §1.72 and the purpose stated in 37 C.F.R. §1.72(b) “to enable the United States Patent and Trademark Office and the public generally to determine quickly from a cursory inspection the nature and gist of the technical disclosure.” Therefore, the Abstract of this disclosure is not intended to be used to construe the scope of the claims or to limit the scope of subject matter that is disclosed herein. Moreover, any headings that may be employed herein are also not intended to be used to construe the scope of the claims or to limit the scope of the subject matter that is disclosed herein.

Generally, the devices and methods of the present disclosure employ lateral flow assay techniques and matrices capable of bibulous and/or non-bibulous lateral flow as generally described in U.S. Pat. Nos. 5,424,193, 4,943,522; 4,861,711; 4,857,453; 4,855,240; 4,775,636; 4,703,017; 4,361,537; 4,235,601; 4,168,146; 4,094,647; and 7,144,742; each of which is incorporated herein by reference.

### I. DEFINITIONS

As used herein, the term “analyte” means a compound or composition to be measured and that is capable of binding to a receptor.

As used herein, the term “antigen” means any compound capable of binding to an antibody.

As used herein, the term “antibody” means an immunoglobulin having an area on its surface or in a cavity that specifically binds to and is thereby defined as complementary with a particular spatial and polar organization of another molecule. The antibody can be polyclonal or monoclonal. Antibodies may include a complete immunoglobulin or fragments thereof, which immunoglobulins include the various classes and isotypes, such as IgA (IgA1 and IgA2), IgD, IgE, IgM, and IgG (IgG1, IgG2, IgG3, and IgG4), etc. Fragments thereof may include Fab, Fv and F(ab′)₂, Fab′, and the like. Antibodies may also include chimeric antibodies made by recombinant methods.

As used herein, the term “antibody for the analyte” means an antibody specific to, or that has a binding affinity for, a particular analyte.

As used herein, the term “lateral flow matrix” means a bibulous or non-bibulous matrix capable of lateral flow.

As used herein, the term “receptor” means any compound or composition capable of recognizing a particular spatial or polar orientation of a molecule. Examples of receptors include, but are not limited to, antibodies, enzymes, nucleic acids, and proteins.

As used herein, a “labeled receptor” means a receptor that is conjugated to or otherwise connected with a detectable reagent or label (e.g., colloidal gold or particulate latex).

As used herein, an “unlabeled receptor” means a receptor that is not conjugated or otherwise connected to a detectable label.

### II. LATERAL FLOW DEVICE

In one aspect, a device for measuring an amount of an analyte in a sample is provided. The device comprises a lateral flow matrix that defines a flow path. The flow path comprises, in series: a sample receiving zone; a labeling zone comprising an unlabeled receptor and a labeled receptor, where the unlabeled receptor is located downstream of the labeled receptor and separated by a distance; and two serially oriented capture zones that are capable of providing visual quantitation of the amount of the analyte in the sample.

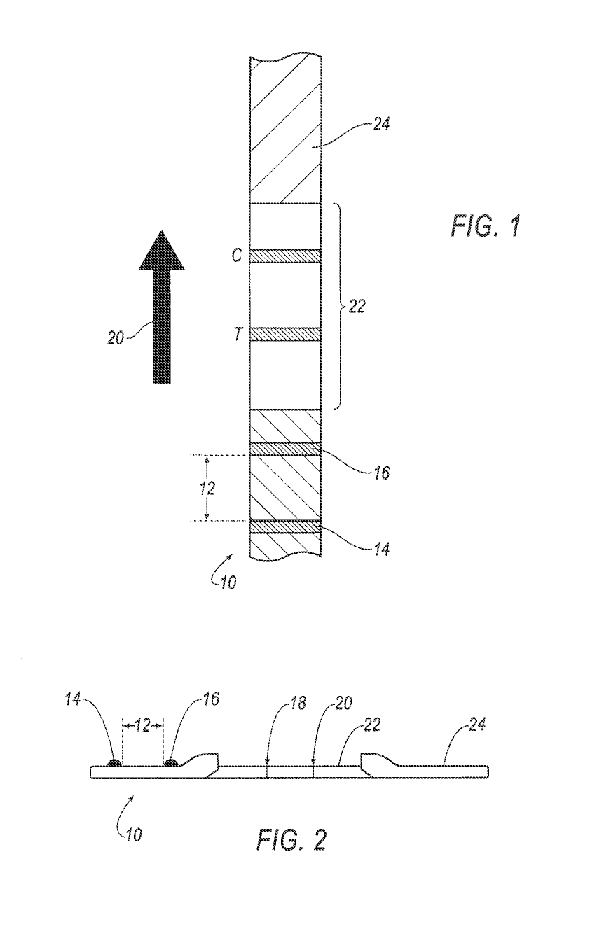

As shown in FIGS. 1 and 2, a lateral flow device for detecting an analyte in a sample may include:

A sample pad 10, which may comprise a compressed material, such as cellulose, that is capable of absorbing a biological fluid and acting as a prefilter to remove coarse contaminants, such as hair, dirt, etc. Sample pad 10 is sized to absorb a fixed amount of sample required to complete the assay. This compressed material, when expanded upon wetting with a sample, causes sufficient pressure to drive capillary flow in a direction of flow 20. The sample pad 10 may further include a labeled receptor region 14 that is separated by a distance 12 from the unlabeled receptor region 16. The sample pad 10 may overlap the cellulosic membrane material 22 by about 1 to 10 mm such that, when an aqueous sample, such as milk, is added to sample pad 10, the sample flows onto the cellulosic membrane material 22.

The cellulosic membrane material 22 may be formed of nitrocellulose, nylon, polyethylene or another suitable material. In cellulosic membrane material 22, the analyte representative drug is attached with a high specific ratio to a carrier, e.g., a protein such as BSA, IgG, or Protein A. The cellulosic membrane material 22 includes test zone 18 sprayed in a line using a suitable spraying instrument. The purpose of the test zone is to capture unreacted binding protein/probe complex for viewing or measurement. Test zone 18 consists of an analyte of detection; that is, the analyte or a member of the analyte family attached to a carrier protein, that is, BSA, IgG, KLH, suspended in a 5 to 100 mM buffer solution (such as phosphate or buffer base) at a pH range of 3-10. Total protein concentration of the antibody solution ranges from 0.2 to 100 mg/ml. The analyte-carrier, dissolved in a buffer solution, e.g., 10 mM phosphate buffer, pH 6.9 containing sugar, such as trehalose or other additives, or 0.1 M sodium bicarbonate containing sugar, such as trehalose or other additives, is sprayed as a line on the stationary-phase membrane. Tentacle immobilization of analyte conjugate to a multiple binding site carrier, such as Protein A or latex microspheres, increases stability and binding capacity. Subsequent heat treatment of the membrane further stabilizes the adhesion.

The cellulosic membrane material 22 also comprises a control zone 20. The control zone 20 may be sprayed in a line form using a suitable spraying instrument. A purpose of control zone 20 is to capture binding protein/probe complex that has not bound to test zone 18. Control zone 20 can consist of an antibody specific to the binding protein/probe suspended in 5 to 100 mM of a buffer solution (e.g., phosphate) in a pH range of 3 to 10. Total protein concentration of the antibody solution ranges generally from 0.2 to 100 mg/mL.

Finally, an absorbance pad 24 may be an absorbing membrane made of a cellulose, synthetic sponge, or other material. This pad keeps the sample flowing and stops flow at saturation, thus giving the assay time control and reducing background noise. The absorbance pad 24 may retain the reacted sample. The absorbance pad 24 may also overlap the stationary phase 22 by about 1 to 5 mm

A comparison of the control zone 20 to the test zone 18 yields the test result. Typically, if the control zone 20 is darker than the test zone 18, analyte is present at detection level or greater.

In another embodiment, the membranes, such as the cellulosic membrane material 22 and sample pad 10 can be blocked, for example, with mixtures of bovine serum albumin, skim milk, polyethylene glycol, sucrose, trehalose, and amino acids to eliminate nonspecific interactions.

In some embodiments, the unlabeled receptor comprises an antibody having a binding affinity for the analyte in the sample. In certain embodiments, the antibody is sheep anti-tetracycline. In other embodiments, the unlabeled receptor comprises a cross-linked antibody having a binding affinity for the analyte in the sample. In some embodiments, the cross-linked antibody comprises a monoclonal antibody species that is cross-linked to an antibody species. In certain embodiments, the monoclonal antibody species is different from the antibody species (e.g., rabbit and sheep). In an embodiment, the monoclonal antibody species is rabbit IgG and the antibody species is sheep anti-tetracycline. In still other embodiments, the cross-linked antibody comprises a small molecule tag that is cross-linked to an antibody species. In an embodiment, the small molecule tag is histamine-tag and the antibody species is sheep anti-tetracycline. Other suitable small molecule tags include, but are not limited to, digoxin-tag, FLAG-tag, biotin-tag, FITC-tag, HA-tag, bsa-tag, and IgG.

In some embodiments, the labeled receptor is bound to a detectable reagent. In certain embodiments, the labeled receptor is bound to detectable microparticles. In some embodiments, the labeled receptor comprises a labeled antibody. In certain embodiments, the labeled receptor comprises labeled anti-sheep, anti-mouse, anti-rabbit, anti-llama, anti-chicken, and anti-human. In some embodiments, the labeled receptor is anti-sheep gold.

In some embodiments, suitable labeled reagents include, but are not limited to, particulate labels such as colored or non-colored latex beads, erythrocytes, liposomes, dye sols, metallic and non-metallic colloids, stained microorganisms, quantum dots (e.g., nano-crystals), superparamagnetic particles, fluorophores (e.g., Alexa Fluor, PE-Cyanine 5), europium, carbon nanoparticles, and the like. In still other embodiments, non-particulate labels are used. In some embodiments, metallic colloids such as colloidal gold may be used. In other embodiments, non-metallic colloids such as colloidal selenium may be used.

A variety of labeling methods can be used, including visible, colorimetric, chemiluminescent, fluorescent, and other known labeling methods. Such labels include but are not limited to particulate labels such as dyed latex beads, erythrocytes, liposomes, dye sols, metallic and nonmetallic colloids, stained microorganisms, and other such labels known to those skilled in the art. Non-particulate labels such as the target-specific antigen complexes described in U.S. patent application Ser. No. 08/408,441, filed Mar. 16, 1995, can also be used. Suitable labels such as colloidal metals, e.g., gold and dye particles are disclosed in U.S. Pat. Nos. 4,313,734 and 4,373,932, both incorporated by reference. Non-metallic colloids, such as colloidal selenium, tellurium, and sulfur are disclosed in U.S. Pat. No. 4,954,452, incorporated by reference. Dyed microorganisms as labels are disclosed in U.S. Pat. No. 5,424,193, EP 0 074 520 and British Patent No. GB 1,194,256, all incorporated by reference. Dyed latex particles are disclosed in U.S. Pat. No. 4,703,017, incorporated by reference.

In certain embodiments, the labeled receptor comprises a colloidal gold-conjugated antibody species. In some embodiments, the gold-conjugated antibody species comprises gold particles that are in the range of about 10 nm to about 100 nm, about 20 nm to about 80 nm, about 20 nm to about 60 nm, about 30 nm to about 60 nm, about 40 nm to about 60 nm, or about 50 nm.

In certain embodiments, the labeled receptor comprises colored and non-colored latex particles. In some embodiments, the latex particles are in the range of about 20 nm to about 600 nm, about 40 nm to about 400 nm, about 60 nm to about 400 nm, about 80 nm to about 200 nm, or about 100 nm to about 200 nm.

In some embodiments, the analyte is any small molecule in a sample for which a government agency has an established maximum or legal limit. Examples of target analytes include, but are not limited to, antibiotics in food (including milk, meat, fish, and honey), toxins in grains, and drugs of abuse in saliva, blood serum, and hair. In further embodiments, the analyte to be detected may include, but is not limited to, toxins, like aflatoxins, pesticides, such as organophosphates and carbamates; as well as beta-lactams, such as penicillin, ampicillin, amoxicillin, cloxacillin, dicloxacillin, oxacillin, ceftiofur, and cephapirin; tetracyclines, such as chlortetracycline, oxytetracycline, and tetracycline; sulfonamides, such as sulfamethazine, sulfadimethoxine, sulfamerazine, sulfathiazole, and sulfadiazine; macrolides, such as erythromycin, spiramycin, and tylosin; aminoglycosides, such as gentamicin, neomycin, and DH/streptomycin; and others such as dapsone, chloramphenicol, novobiocin, spectinomycin, and trimethoprim, to detect the maximum residue-analyte limits in the sample. Most of the elements for each test are the same except the chemistries of the mobile phase, test zone, and control zone, which are tailored to the specific analyte detection.

In some embodiments, the analyte is an antibiotic commonly found in foodstuffs. In certain embodiments, the analyte is an antibiotic from the group consisting of tetracyclines, beta lactams, quinolones, aminoglycosides, cephalosporins, macrolides, nitrofurans, and sulfonamides. In certain embodiments, the analyte is tetracycline.

In some embodiments, the analyte is a toxin commonly found in foodstuffs. In certain embodiments, the analyte is a toxin from the group consisting of mycotoxins, shellfish toxins, and pesticides.

In certain embodiments, the analyte to be detected is a tetracycline and the device is configured not to detect other analytes. In certain embodiments, the analyte to be detected is a beta-lactam and the device is configured not to detect other analytes. In some embodiments, the analyte to be detected is a quinolone and the device is configured not to detect other analytes. In some embodiments, the analyte to be detected is an aminoglycoside and the device is configured not to detect other analytes. In certain embodiments, the analyte to be detected is a cephalosporin and the device is configured not to detect other analytes. In some embodiments, the analyte to be detected is a macrolide and the device is configured not to detect other analytes. In other embodiments, the analyte to be detected is a nitrofuran and the device is configured not to detect other analytes. In some embodiments, the analyte to be detected is a sulfonamide and the device is configured not to detect other analytes. In some embodiments, the analyte to be detected is a mycotoxin and the device is configured not to detect other analytes. In some embodiments, the analyte to be detected is a shellfish toxin and the device is configured not to detect other analytes. In some embodiments, the analyte to be detected is histamine and the device is configured not to detect other analytes. In other embodiments, the analyte to be detected is a particular controlled substance and the device is configured not to detect other analytes.

In some embodiments, the distance between the labeled receptor and the unlabeled receptor is about 5 mm to about 75 mm, about 5 mm to about 50 mm, about 10 mm to about 50 mm, about 10 mm to about 40 mm, about 15 mm to about 30 mm, about 15 mm to about 25 mm, about 15 mm to about 20 mm, or about 15 mm.

In some embodiments, the device is configured to detect one or more analytes at a sensitivity of about 1 ppb to about 500 ppb. In certain embodiments, the device detects tetracycline at a sensitivity of about 25 ppb to about 200 ppb, about 25 to about 150 ppb, about 30 ppb to about 120 ppb, about 40 ppb to about 100 ppb, about 50 ppb to about 100 ppb, or about 75 to about 100 ppb.

In some embodiments, an increase in the distance between the labeled receptor and the unlabeled receptor affects the sensitivity. In some embodiments, an increase in the distance decreases the sensitivity. In some embodiments, a result that an analyte is present in the sample at or above a threshold level is a positive result.

In some embodiments, one of the two serially oriented capture zones comprises a test zone 18. In some embodiments, one of the two serially oriented capture zones comprises a control zone 20. In some embodiments, the control zone 20 comprises a control binder.

In some embodiments, the lateral flow matrix may comprise one or more bibulous materials, such as the cellulosic membrane material 22. Suitable bibulous materials include, but are not limited to, untreated paper, cellulose, nitrocellulose, polyester, acrylonitrile copolymers, rayon, glass fibers, and the like. In some embodiments, the bibulous material comprises a nitrocellulose material, which includes any nitric acid ester of cellulose. In some embodiments, the pore size of the nitrocellulose material is about 0.5 microns to about 30 microns, about 1 micron to about 20 microns, or about 8 microns to about 15 microns.

In some embodiments, the device may further comprise a solvent for the sample or the analyte. In some embodiments, the solvent is an aqueous solvent. In some embodiments, the aqueous solvent may comprise up to about 40 wt % of a polar organic solvent, including but not limited to oxygen-atom containing solvents of from 1 to 6 carbon atoms. Suitable organic solvents include, but are not limited to, alcohols, ethers, and the like.

In some embodiments, the lateral flow matrix has a pH of about 4 to about 11, about 5 to about 10, or about 6 to about 9. In certain embodiments, the pH is maintained by the use of a suitable buffer, including but not limited to borate, phosphate, carbonate, tris, barbital, and the like.

In some embodiments, the lateral flow matrix may further comprise a non-ionic detergent. In some embodiments, the non-ionic detergent may comprise a polyoxyalkylene compound. In certain embodiments, the concentration of the non-ionic detergent may be about 0.05 to about 0.5 wt % of the solvent.

In certain embodiments, substantially constant temperatures are used for carrying out the assays. The temperatures for the assay and production of a detectable signal will generally be in the range of about 4° C. to about 50° C., about 10° C. to about 40° C., or about 15° C. to about 25° C.

The spatial separation between the zones, and the flow rate characteristics of the cellulosic membrane material 22, can be selected to allow adequate reaction times during which the necessary specific binding can occur, and to allow the labeled and unlabeled receptors in the first zone to dissolve or disperse in the liquid sample and migrate through the carrier. Further control over these parameters can be achieved by the incorporation of viscosity modifiers (e.g., sugars and modified celluloses) in the sample to slow down the reagent migration.

Reagents may be applied to the cellulosic membrane material 22 in a variety of ways. Various “printing” techniques have previously been proposed for application of liquid reagents to carriers, e.g. micro-syringes, pens using metered pumps, direct printing and ink-jet printing, and any of these techniques can be used in the present context. To facilitate manufacture, the carrier (e.g. sheet) can be treated with the reagents and then subdivided into smaller portions (e.g. small narrow strips each embodying the required reagent-containing zones) to provide a plurality of identical carrier units.

### III. ANALYTE MEASUREMENT

The present disclosure is a competitive format assay. A sample comprising an analyte is contacted with a lateral flow matrix device, wherein the analyte binds to at least one of an unlabeled or a labeled receptor to form one or more analyte-receptor complexes. The sample is then allowed to traverse a matrix capable of lateral flow (also termed “a lateral flow matrix”), past a series of spatially separated capture zones located on the matrix. The sample flows sequentially past the series of capture zones and a quantity of the receptor binder bound to at least one of the labeled or unlabeled receptors is detected.

In an aspect, a method for measuring an amount of an analyte in a sample is provided. The method comprises the steps of: providing a lateral flow matrix device comprising an unlabeled receptor and a labeled receptor, the unlabeled receptor located downstream of the labeled receptor and separated by a distance; contacting the sample to the lateral flow matrix device, wherein the analyte binds to at least one of the unlabeled receptor or the labeled receptor to form one or more analyte-receptor complexes; allowing the sample to come into contact with a receptor binder on a solid support, wherein the receptor binder binds to the at least one of the unlabeled or the labeled receptors but does not bind to the one or more analyte-receptor complexes; and detecting a quantity of the receptor binder bound to the at least one of the unlabeled or labeled receptors as an inverse indication of the amount of the analyte in the sample at or above a predetermined threshold level.

In some embodiments, the unlabeled receptor comprises an antibody having a binding affinity for the analyte in the sample. In certain embodiments, the antibody is sheep anti-tetracycline. In other embodiments, the unlabeled receptor comprises a cross-linked antibody having a binding affinity for the analyte in the sample. In some embodiments, the cross-linked antibody comprises a monoclonal antibody species that is cross-linked to an antibody species. In certain embodiments, the monoclonal antibody species is different from the antibody species (e.g., rabbit and sheep). In an embodiment, the monoclonal antibody species is rabbit IgG and the antibody species is sheep anti-tetracycline. In still other embodiments, the cross-linked antibody comprises a small molecule tag that is cross-linked to an antibody species. In an embodiment, the small molecule tag is histamine and the antibody species is sheep anti-tetracycline.

In some embodiments, the labeled receptor is bound to a detectable reagent. In certain embodiments, the labeled receptor is bound to detectable microparticles. In some embodiments, the labeled receptor comprises a labeled antibody. In certain embodiments, the labeled receptor comprises labeled anti-sheep. In some embodiments, the labeled receptor comprises anti-sheep gold.

In some embodiments, suitable labeled reagents include, but are not limited to, particulate labels such as colored or non-colored latex beads, erythrocytes, liposomes, dye sols, metallic and non-metallic colloids, stained microorganisms, quantum dots (e.g., nano-crystals), superparamagnetic particles, fluorophores (e.g., Alexa Fluor, PE-Cyanine 5), europium, carbon nanoparticles, and the like. In still other embodiments, non-particulate labels are used. In some embodiments, metallic colloids such as colloidal gold may be used. In other embodiments, non-metallic colloids such as colloidal selenium may be used.

A variety of labeling methods can be used, including visible, colorimetric, chemiluminescent, fluorescent, and other known labeling methods. Such labels include but are not limited to particulate labels such as dyed latex beads, erythrocytes, liposomes, dye sols, metallic and nonmetallic colloids, stained microorganisms, and other such labels known to those skilled in the art. Non-particulate labels such as the target-specific antigen complexes described in U.S. patent application Ser. No. 08/408,441, filed Mar. 16, 1995, can also be used. Suitable labels such as colloidal metals, e.g., gold and dye particles are disclosed in U.S. Pat. Nos. 4,313,734 and 4,373,932, both incorporated by reference. Non-metallic colloids, such as colloidal selenium, tellurium, and sulfur are disclosed in U.S. Pat. No. 4,954,452, incorporated by reference. Dyed microorganisms as labels are disclosed in U.S. Pat. No. 5,424,193, EP 0 074 520 and British Patent No. GB 1,194,256, all incorporated by reference. Dyed latex particles are disclosed in U.S. Pat. No. 4,703,017, incorporated by reference.

In certain embodiments, the labeled receptor comprises a colloidal gold-conjugated antibody species. In some embodiments, the gold-conjugated antibody species comprises gold particles that are in the range of about 10 nm to about 100 nm, about 20 nm to about 80 nm, about 20 nm to about 60 nm, about 30 nm to about 60 nm, about 40 nm to about 60 nm, or about 50 nm.

In certain embodiments, the labeled receptor comprises colored and non-colored latex particles. In some embodiments, the latex particles are in the range of about 20 nm to about 600 nm, about 40 nm to about 400 nm, about 60 nm to about 400 nm, about 80 nm to about 200 nm, or about 100 nm to about 200 nm.

In some embodiments, the analyte is any small molecule in a sample for which a government agency has an established maximum or legal limit. Examples of target analytes include, but are not limited to, antibiotics in food (including milk, meat, fish, and honey), toxins in grains, and drugs of abuse in saliva, blood serum, and hair. In further embodiments, the analyte to be detected may include, but is not limited to, toxins, like aflatoxins, pesticides, such as organophosphates and carbamates; as well as beta-lactams, such as penicillin, ampicillin, amoxicillin, cloxacillin, dicloxacillin, oxacillin, ceftiofur, and cephapirin; tetracyclines, such as chlortetracycline, oxytetracycline, and tetracycline; sulfonamides, such as sulfamethazine, sulfadimethoxine, sulfamerazine, sulfathiazole, and sulfadiazine; macrolides, such as erythromycin, spiramycin, and tylosin; aminoglycosides, such as gentamicin, neomycin, and DH/streptomycin; and others such as dapsone, chloramphenicol, novobiocin, spectinomycin, and trimethoprim, to detect the maximum residue-analyte limits in the sample. Most of the elements for each test are the same except the chemistries of the mobile phase, test zone, and control zone, which are tailored to the specific analyte detection.

In some embodiments, the analyte is an antibiotic commonly found in foodstuffs. In certain embodiments, the analyte is an antibiotic from the group consisting of tetracyclines, beta lactams, quinolones, aminoglycosides, cephalosporins, macrolides, nitrofurans, and sulfonamides. In certain embodiments, the analyte is tetracycline.

In some embodiments, the analyte is a toxin commonly found in foodstuffs. In certain embodiments, the analyte is a toxin from the group consisting of mycotoxins, shellfish toxins, and pesticides.

In certain embodiments, the analyte to be detected is a tetracycline and the device is configured not to detect other analytes. In certain embodiments, the analyte to be detected is a beta-lactam and the device is configured not to detect other analytes. In some embodiments, the analyte to be detected is a quinolone and the device is configured not to detect other analytes. In some embodiments, the analyte to be detected is an aminoglycoside and the device is configured not to detect other analytes. In certain embodiments, the analyte to be detected is a cephalosporin and the device is configured not to detect other analytes. In some embodiments, the analyte to be detected is a macrolide and the device is configured not to detect other analytes. In other embodiments, the analyte to be detected is a nitrofuran and the device is configured not to detect other analytes. In some embodiments, the analyte to be detected is a sulfonamide and the device is configured not to detect other analytes. In some embodiments, the analyte to be detected is a mycotoxin and the device is configured not to detect other analytes. In some embodiments, the analyte to be detected is a shellfish toxin and the device is configured not to detect other analytes. In some embodiments, the analyte to be detected is histamine and the device is configured not to detect other analytes. In other embodiments, the analyte to be detected is a particular controlled substance and the device is configured not to detect other analytes.

In some embodiments, the distance between the labeled receptor and the unlabeled receptor is about 5 mm to about 75 mm, about 5 mm to about 50 mm, about 10 mm to about 50 mm, about 10 mm to about 40 mm, about 15 mm to about 30 mm, about 15 mm to about 25 mm, about 15 mm to about 20 mm, or about 15 mm.

In some embodiments, the device is configured to detect one or more analytes at a sensitivity of about 1 ppb to about 500 ppb. In certain embodiments, the device detects tetracycline at a sensitivity of about 25 ppb to about 200 ppb, about 25 to about 150 ppb, about 30 ppb to about 120 ppb, about 40 ppb to about 100 ppb, about 50 ppb to about 100 ppb, or about 75 to about 100 ppb.

In some embodiments, an increase in the distance between the labeled receptor and the unlabeled receptor affects the sensitivity. In some embodiments, an increase in the distance decreases the sensitivity. In some embodiments, a result that an analyte is present in the sample at or above a threshold level is a positive result.

In some embodiments, one of the two serially oriented capture zones comprises a test zone 18. In some embodiments, one of the two serially oriented capture zones comprises a control zone 20. In some embodiments, the control zone 20 comprises a control binder.

In some embodiments, the lateral flow matrix further comprises a cellulosic membrane material 22. In some embodiments, the cellulosic membrane material 22 may comprise one or more bibulous materials. Suitable bibulous materials include, but are not limited to, untreated paper, cellulose, nitrocellulose, polyester, acrylonitrile copolymers, rayon, glass fibers, and the like. In some embodiments, the bibulous material comprises a nitrocellulose material, which includes any nitric acid ester of cellulose. In some embodiments, the pore size of the nitrocellulose material is about 0.5 microns to about 30 microns, about 1 micron to about 20 microns, or about 8 microns to about 15 microns.

In some embodiments, the device may further comprise a solvent for the sample or the analyte. In some embodiments, the solvent is an aqueous solvent. In some embodiments, the aqueous solvent may comprise up to about 40 wt % of a polar organic solvent, including but not limited to oxygen-atom containing solvents of from 1 to 6 carbon atoms. Suitable organic solvents include, but are not limited to, alcohols, ethers, and the like.

In some embodiments, the lateral flow matrix has a pH of about 4 to about 11, about 5 to about 10, or about 6 to about 9. In certain embodiments, the pH is maintained by the use of a suitable buffer, including but not limited to borate, phosphate, carbonate, tris, barbital, and the like.

In some embodiments, the lateral flow matrix may further comprise a non-ionic detergent. In some embodiments, the non-ionic detergent may comprise a polyoxyalkylene compound. In certain embodiments, the concentration of the non-ionic detergent may be about 0.05 to about 0.5 wt % of the solvent.

In certain embodiments, substantially constant temperatures are used for carrying out the assays. The temperatures for the assay and production of a detectable signal will generally be in the range of about 4° C. to about 50° C., about 10° C. to about 40° C., or about 15° C. to about 25° C.

The spatial separation between the zones, and the flow rate characteristics of the cellulosic membrane material 22, can be selected to allow adequate reaction times during which the necessary specific binding can occur, and to allow the labeled and unlabeled receptors in the first zone to dissolve or disperse in the liquid sample and migrate through the carrier. Further control over these parameters can be achieved by the incorporation of viscosity modifiers (e.g., sugars and modified celluloses) in the sample to slow down the reagent migration.

Reagents may be applied to the cellulosic membrane material 22 in a variety of ways. Various “printing” techniques have previously been proposed for application of liquid reagents to carriers, e.g. micro-syringes, pens using metered pumps, direct printing and ink-jet printing, and any of these techniques can be used in the present context. To facilitate manufacture, the carrier (e.g. sheet) can be treated with the reagents and then subdivided into smaller portions (e.g. small narrow strips each embodying the required reagent-containing zones) to provide a plurality of identical carrier units.

In some embodiments, an increase in the distance 12 between the labeled receptor and the unlabeled receptor affects the sensitivity. In some embodiments, an increase in the distance 12 decreases the sensitivity. In some embodiments, the predetermined threshold level is inversely correlated to the distance 12 between the labeled receptor and the unlabeled receptor.

In some embodiments, the lateral flow matrix device further comprises a control zone 20. In some embodiments, the control zone 20 comprises a control binder characterized in that it binds both to the at least one of the unlabeled or labeled receptors and to the one or more analyte-receptor complexes. In some embodiments, the method further includes detecting a quantity of the at least one of the unlabeled or the labeled receptors and the one or more analyte-receptor complexes bound to the control binder.

In some embodiments, the lateral flow matrix device further comprises a test zone 18. In some embodiments, the test zone 18 comprises the quantity of the receptor binder bound to the at least one unlabeled or labeled receptors. In some embodiments, the step of detecting further comprises comparing a first signal obtained from the test zone 18 with a second signal obtained from the control zone 20, the method configured to provide a positive result when the analyte is present at or above the predetermined threshold level, wherein the positive result is indicated by a more intense second signal as compared to the first signal.

### IV. ANALYTE MEASUREMENT KITS

In another aspect, a kit for measuring an amount of an analyte in a sample is provided. In some embodiments, the kit comprises the devices described herein for performing the methods described herein with instructions for performing the method and interpreting the assay results. The kits are designed for testing antibiotics, toxins, and pesticides in food or environmental samples in the field, or in the lab.

In some embodiments, a kit for detecting the presence of a predetermined threshold amount of an analyte in a sample is provided. The kit includes a container that comprises a lateral flow matrix which defines a flow path and which comprises in series: a sample receiving zone; a labeling zone comprising an unlabeled receptor and a labeled receptor, the unlabeled receptor located downstream of the labeled receptor and separated by a distance; and two serially oriented capture zones capable of providing quantification of the amount of analyte in the sample, wherein a result that the analyte is present in the sample at or above the predetermined threshold amount is a positive result.

In some embodiments, the kits further comprise a housing. The housing may be configured to allow for addition of a sample, either by dripping, pouring, or pipetting. The housing may be constructed of a flexible or hard material, such as polystyrene, polypropylene, or polyethylene.

In other embodiments, the kits may further comprise an incubator. The incubator may be incorporated directly into the housing or may be an external unit configured to attach to and then surround the housing.

### V. EXAMPLES

The following examples are offered by way of illustration, not by way of limitation.

### Example 1

Effect of Antibody Amount on Test Line Signal Intensity

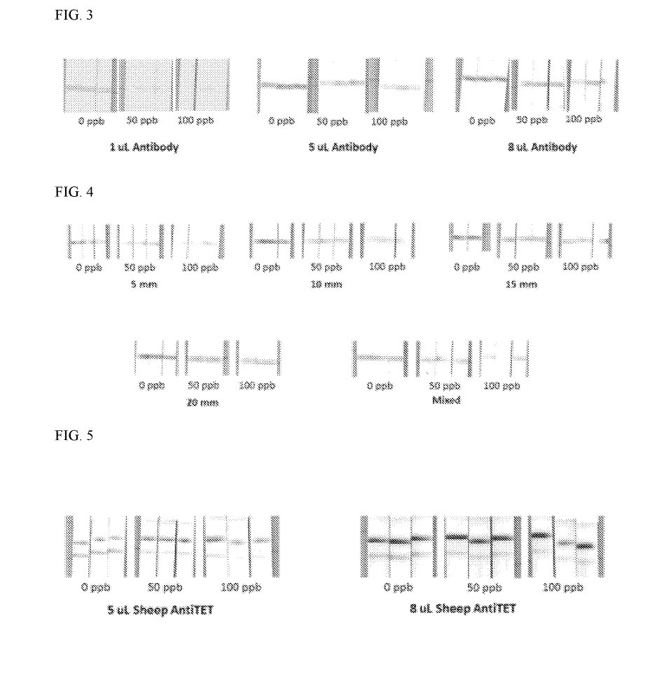

In this example, different amounts of sheep anti-tetracycline unlabeled receptor was used with a constant amount of gold-conjugated labeled receptor and a constant amount of test line material. The labeled receptor was prepared using 4 μL of a 4 OD colloidal gold compound diluted in 4D run buffer (Neogen). This labeled receptor was spotted onto a Standard 17 (Whatman/GE) sample pad. The test line was prepared using 2.5 mg/mL of tet-bsa diluted in 0.5% trehalose (Sigma) with 1× PBS (Neogen). Individual pads were then spotted with 0, 1, 2, 3, 4, 5, 6, 7, 8, 9, or 10 μL of a solution of sheep anti-tetracycline (Randox). The unlabeled sheep anti tetracycline was prepared by diluting the stock antibody (Randox) with 4D run buffer (50 mg/mL bsa, 0.1M NaHPO₄, 1% pluronic F98, 0.1% azide). The unlabeled receptor was spotted ˜25 mm downstream of the gold-conjugated labeled receptor (donkey anti sheep conjugated to colloidal gold). The spotted pads were then dried for 2 minutes at 44° C. Three lateral flow assays were run at 47.5° C. for 5 minutes using each quantity of the unlabeled receptor, with each run conducted in the presence of each of three analytes: raw milk with 0 ppb tetracycline, 50 ppb oxy tetracycline milk, and 100 ppb oxy tetracycline milk. The assay results for the 1 μL sample, the 5 μL sample, and the 8 μL sample are shown in FIG. 3. The signal intensity was found to increase concomitantly with the increase in antibody volume. As a result, the sensitivity decreased. Surprisingly, a negative result was observed using only 1 μL of the antibody solution when in the presence of 50 ppb of oxy tetracycline.

### Example 2

Effect of Spacing Between Labeled and Unlabeled Receptors on Test Line Signal

In this example, a constant amount of sheep anti-tetracycline unlabeled receptor was used with a constant amount of gold-conjugated labeled receptor and a constant amount of test line material. The variable was the distance between the unlabeled and labeled receptors. The labeled receptor was prepared using 4 μL of a 4 OD colloidal gold compound diluted in 4D run buffer. This labeled receptor was spotted onto a Standard 17 sample pad, 5 mm from the bottom of the pad. The test line was prepared using 2.5 mg/mL of tet-bsa diluted in 0.5% trehalose with 1× PBS. Sets of pads were then prepared, with the unlabeled receptor spotted downstream of the gold-conjugated labeled receptor. A 5 μL volume of antibody solution was spotted onto each set of pads. The antibody solutions were spotted at distances of 5 mm, 10 mm, 15 mm, and 20 mm upstream from the labeled receptor. An additional set of pads was prepared where the antibody solution was spotted directly onto the labeled receptor. The spotted pads were then dried for 2 minutes at 44° C. Three lateral flow assays were run for each set of pads at 47.5° C. for 5 minutes using each of three analytes: raw milk with 0 ppm tetracycline, 50 ppb oxy tetracycline milk, and 100 ppb oxy tetracycline milk. The results are shown in FIG. 4. Surprisingly, a greater distance between the labeled and unlabeled spots resulted in a reduced observed sensitivity.

### Example 3

Quantification of Test Line Signal Based on Amount of Unlabeled Receptor

In this example, different amounts of sheep anti-tetracycline unlabeled receptor was used with a constant amount of gold-conjugated labeled receptor (chicken IgY colloidal gold; BBI) and a constant amount of test line material (goat anti chicken; BBI). Control lines were added to enable quantification of the intensity of the test line signal using an Accuscan Pro spectrometer (Axxin), although the control lines were not optimized for signal intensity. The labeled receptor was prepared using 4 μL of a 4 OD colloidal gold compound diluted in 4D run buffer. This labeled receptor was spotted onto a Standard 17 pad, 5 mm from the bottom of the pad. The test line was prepared using 2.5 mg/mL of tet-bsa diluted in 0.5% trehalose with 1× PBS. Sets of pads were then prepared, with the unlabeled receptor spotted ˜25 mm downstream of the gold-conjugated labeled receptor. Two sets of pads were prepared, one with 5 μL and a second with 8 μL of a solution of antibody spotted onto each pad. The antibody solutions were spotted ˜25 mm upstream from the labeled receptor. The spotted pads were then dried for 2 minutes at 44° C. Three lateral flow assays were run for each set of pads at 47.5° C. for 5 minutes using each of three analytes: raw milk with 0 ppm tetracycline, 50 ppb oxy tetracycline milk, and 100 ppb oxy tetracycline milk. The lateral flow assays were performed three times for each analyte and the observed signal intensity was averaged over the three runs. The results are shown in FIG. 5 and summarized in Table 1. The data showed that the 8 μL unlabeled sample resulted in a significant decrease in observed signal intensity, although a decrease in the percent of inhibition at 50 ppb was also noted. The results suggested that the amount of unlabeled receptor might be the limiting reagent, as there was an insufficient quantity to capture all of the antibody.

TABLE 1

Amount of Antibody

| | 5 μL | 8 μL |
|---|---|---|
| Negative Average | 7124 | 4737 |
| 50 ppb Average | 2541 | 3351 |
| 100 ppb Average | 1532 | 2467 |

### Example 4

Quantification of Test Line Signal Based on Amount of Unlabeled Receptor

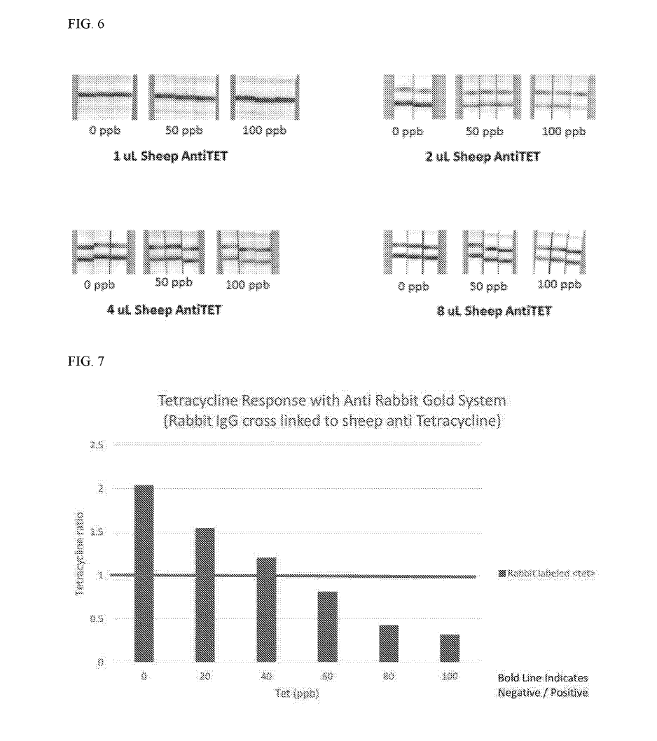

In this example, different amounts of sheep anti-tetracycline unlabeled receptor were used with a constant amount of gold-conjugated labeled receptor and a constant amount of test line material. Control lines were added to enable quantification of the intensity of the test line signal using an Accuscan Pro spectrometer, although the control lines were not optimized for signal intensity. The labeled receptor was prepared using 4 μL of a 6 OD colloidal gold compound diluted in 4D run buffer. The labeled receptor was spotted onto a Standard 17 sample pad, 5 mm from the bottom of the pad. The test line was prepared using 2.5 mg/mL of tet-bsa diluted in 0.5% trehalose with 1× PBS. Sets of pads were then prepared, with the unlabeled receptor spotted downstream of the gold-conjugated labeled receptor. Four sets of pads were prepared, with 1 μL, 2 μL, 4 μL, and 8 μL volumes of a solution of antibody spotted onto each pad. The antibody solutions were spotted ˜25 mm upstream from the labeled receptor. The spotted pads were then dried for 2 minutes at 44° C. Three lateral flow assays were run for each set of pads at 47.5° C. for 5 minutes using each of three analytes: raw milk with 0 ppm tetracycline, 50 ppb oxy tetracycline milk, and 100 ppb oxy tetracycline milk. The observed signal intensity was averaged over three runs. The results are shown in FIG. 6 and summarized in Table 2. Surprisingly, the results showed that increasing the amount of labeled receptor also decreased the observed sensitivity.

TABLE 2

Amount of Antibody

| | 1 μL | 2 μL | 4 μL | 8 μL |
|---|---|---|---|---|
| Negative Average | 2037 | 22727 | 23559 | 25204 |
| 50 ppb Average | 2038 | 11915 | 16811 | 20359 |
| 100 ppb Average | 2042 | 7895 | 11958 | 16465 |

### Example 5

Quantification of Test Line Signal Based on Amount of Unlabeled Receptor

In this example, different amounts of sheep anti-tetracycline unlabeled receptor were used with a constant amount of gold-conjugated labeled receptor and a constant amount of test line material. Control lines were added to enable quantification of the intensity of the test line signal using an Accuscan Pro spectrometer, although the control lines were not optimized for signal intensity. The labeled receptor was prepared using 4 μL of a 5 OD colloidal gold compound diluted in 4D run buffer. The labeled receptor was spotted onto a Standard 17 sample pad, 5 mm from the bottom of the pad. The test line was prepared using 2.5 mg/mL of tet-bsa diluted in 0.5% trehalose with 1× PBS. Sets of pads were then prepared, with the unlabeled receptor spotted downstream of the gold-conjugated labeled receptor. Two sets of pads were prepared, using 1 μL and 2 μL volumes of a solution of antibody spotted onto each pad. The antibody solutions were spotted ˜25 mm upstream from the labeled receptor. The spotted pads were then dried for 2 minutes at 44° C. Three lateral flow assays were run for each set of pads at 47.5° C. for 5 minutes using each of three analytes: raw milk with 0 ppm tetracycline, 50 ppb oxy tetracycline milk, and 100 ppb oxy tetracycline milk. The lateral flow assays were performed three times for each analyte and the observed signal intensity was averaged over the three runs. The results are summarized in Table 3. The data suggested that increasing the amount of antibody resulted in a decrease in the sensitivity of the system without any decrease in negative test line intensity. The results suggested that if an additional amount of antibody is required to further decrease sensitivity, more labeled receptor might be added to capture additional material.

Additionally, the results showed that a ratio-based measurement was possible. Normal negative/positive results were based on a test/control line intensity difference of 1.0. Thus, if a normal control line intensity was 4000 units, then signals from samples containing as little as 100 ppb of analyte could be deemed negative results. This suggested that the sensitivity could be adjusted to any desirable level based on the concentration of analyte to be detected.

TABLE 3

Amount of Antibody

| | 1 μL | 2 μL |
|---|---|---|
| Negative Average | 17406.38 | 21201.5 |
| 50 ppb Average | 6801.667 | 12710.17 |
| 100 ppb Average | 4372.5 | 8757.667 |

### Example 6

Use of a Cross-Linked Unlabeled Receptor to Avoid Species-Specific Interactions

The utility of a cross-linked primary (unlabeled) antibody system was explored in an attempt to avoid species-specific interactions that could cause a reduction in signal intensity. In this example, the primary antibody was cross-linked with rabbit IgG. The labeled receptor was rabbit IgG conjugated to colloidal gold.

Method for cross-linking antibodies: Sheep anti-tet and rabbit IgG were reacted at a 3:1 ratio using a sulfo-SMCC cross linker as well as Traut's reagent (https://www.piercenet.com/instructions/2160414.pdf) to thiolate the amine groups on the rabbit IgG. Traut's reagent was reacted with rabbit IgG at a 10-molar excess ratio for 1 hour in 100/150/9.0 PBS with 5 mM EDTA and then desalted using a Sephadex G-25 desalting column into 100/150/7.0 PBS with 5 mM EDTA. Simultaneously, sheep anti-tet was reacted with a 20-molar excess of sulfo SMCC for 1 hour in 100/150/7.4 PBS and desalted using a Sephadex G-25 desalting column into 100/150/7.0 PBS with 5 mM EDTA. Once both the rabbit IgG and sheep anti-tet were free of unreacted Traut's reagent and SMCC, respectively, 3 mg of thiolated rabbit IgG was reacted with 1 mg of sheep anti-tet for 2 hours at ambient temperature in 100/150/7.0 PBS with 5 mM EDTA. The reaction product was purified by either using a Sephadex G-25 desalting column into 20/150/7.2 PBS with 5 mM EDTA or dialyzed against 20/150/7.2 PBS for four cycles at three hours per cycle. All reagents were obtained from Pierce (Thermo Scientific or Sigma). The absorbance at 280 nm was measured to determine the concentration of the protein in the resulting sample.

Assay format: A prototype betastar Combo S device was used, measuring 9 cm in height and having a width of 0.41 cm. The device contains several laminated materials, including: (a) a 28 mm wide nitrocellulose membrane which was placed on the adhesive backing card 25 mm from the bottom of the card; (2) a 27 mm wide Standard 17 sample pad which was placed at the bottom of the backing card overlapping the nitrocellulose membrane; (3) an absorbent wicking pad which was placed above the nitrocellulose and overlapping on top of it; (4) a thin translucent over laminate plastic which covered the nitrocellulose/sample pad material.

The nitrocellulose membrane was prepared by adding: (a) 3.0 mg/mL of the tetracycline-protein conjugate unlabeled receptor containing 0.05% trehalose in 20/150/7.4 PBS; (b) a tetracycline test line was striped onto the nitrocellulose at 1 uL/cm; (c) a control line was also striped at 1.0 mg/mL under the same conditions; (d) the tetracycline conjugate was placed 8 mm from the bottom of the nitrocellulose and the control line was placed 20 mm from the bottom of the nitrocellulose.

The Standard 17 sample pad was prepared by: (a) spraying a gold solution onto the pad at 3 uL/cm, where the labeled receptor solution contained 5 OD goat anti-rabbit gold, 1 OD anti-control line gold, 5% sucrose, 1% surfactant, and 2 mM Borax pH 9.0 buffer; (b) the gold was sprayed 5 mm from the bottom of the sample pad; (c) an antibody line containing 280 ng of the cross linked, unlabeled antibody receptor, 2% BSA, 5% sucrose, and 1% surfactant in 20/150/7.2 PBS; (d) the unlabeled receptor was sprayed 25 mm from the bottom of the pad at 4 μL/cm, resulting in a distance of 20 mm between the labeled and the unlabeled receptors.

The reagents were then dried at 37° C. for approximately 5 minutes. After the device was assembled, the cards were cut to an individual size of 0.41 cm for testing. The lateral assays were performed over the course of 5-10 min at 47.5° C.

The results are shown in FIGS. 7-9. FIG. 7 shows that the system was capable of detecting between 50-60 ppb of tetracycline in milk. As shown in FIG. 7, the “tetracycline ratio” represents the area output of the tetracycline test line divided by the area output of the control line. Measurements were made using the Accuscan pro.

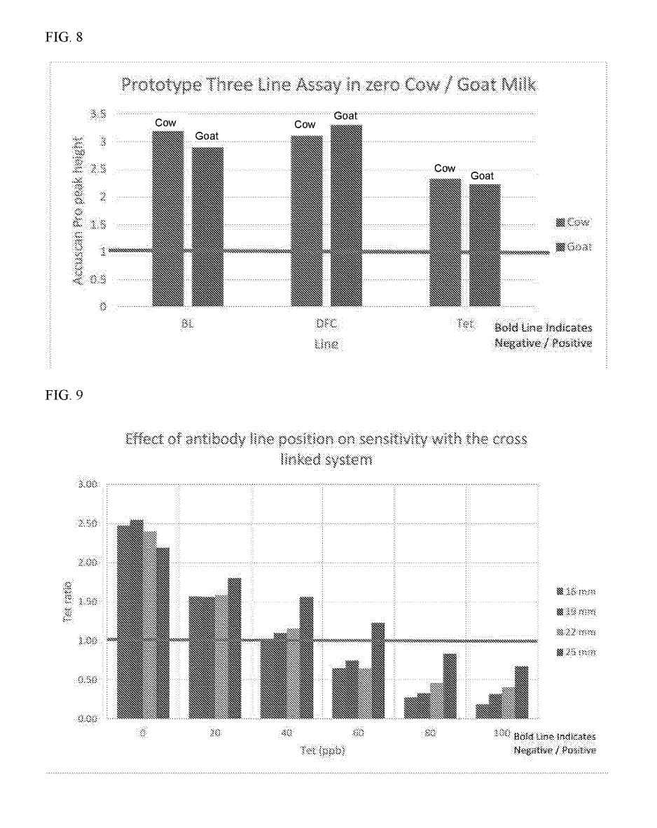

FIG. 8 demonstrates that the described system is capable of being used with both cow milk and goat milk. The data demonstrates how the assay was unaffected by goat milk, which in a previous system (anti-sheep/sheep anti-tet) would have influenced the results greatly. The data was obtained using a prototype BetaStar Combo S test configured to detect beta-lactams, des-ceft, and tetracyclines. The data shows that by using the cross-linked system, goat milk can be tested on the same system as is used for cow milk.

FIG. 9 demonstrates that the distance between the labeled and unlabeled receptors affects the detection sensitivity of the lateral flow assay system. As shown in the graph, the distance between receptors can be adjusted based on the desired level of detection sensitivity. As shown in FIG. 9, the y-axis represents the tetracycline ratio, which is the measured area output of the tetracycline test line divided by the area output of the control line. The greater the ratio, the greater the signal intensity of the test line. From FIG. 7, a trend is observed such that as the distance increases the ratio also increases across the dose response curve. A ratio of 1.0 determines negative or positive, so increasing the distance made the assay less sensitive.

### Example 7

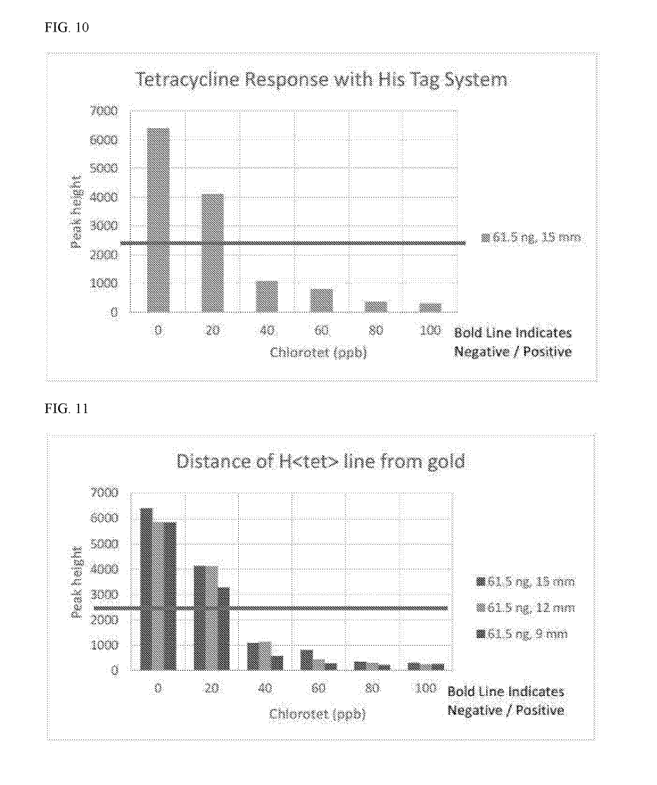

The utility of a small molecule antibody tag detection system was explored to desensitize the assay and remove cross-reactivity from species-specific interactions. In this example, sheep anti-tet was conjugated to histamine using standard methods in the art. The distance between the labeled and unlabeled receptor was varied, and an antibody directed against histamine that had been conjugated to colloidal gold was used as the labeled receptor. The assay protocol was the same as used in Example 6. The results are shown in FIG. 10. As seen in FIG. 10, the system has been desensitized to above 20 ppb tetracycline. FIG. 11 shows a comparison of how the distance between the labeled and unlabeled receptors changes the sensitivity of the immunochromatographic assay. As seen in FIG. 11, increasing the distance between the labeled and the unlabeled receptors decreases the sensitivity of the assay.

Histamine conjugation protocol: A carbonate buffer was prepared using 10% (1M) sodium carbonate in Milli Q H₂O. A bicarbonate buffer was prepared using 8.5% (1M) sodium bicarbonate in Milli Q H₂O. The carbonate solution was slowly added to the bicarbonate solution until the pH is equal to 9.0. The solution was diluted 10:1 for a final 0.1M buffer solution. A 50 mM phosphate buffer at pH 6.75 was also prepared and a 0.5 M monobasic phosphate buffer at pH<6 was also prepared. The tet solution was prepared by placing 2.19 mL of a tet stock solution into a 15 mL sample size ultracentrifuge cartridge. The tet stock solution was then diluted to the maximum volume of the cartridge with the carbonate buffer. The cartridge was placed into a centrifuge and spun to concentrate the solution. The solution was dilute again with the carbonate buffer to the maximum volume of the cartridge and centrifuged again. The resulting material was diluted to 500 μL to make a roughly 10 mg/mL solution in carbonate buffer. The activated histamine solution was made by preparing a 2 mg/mL solution of histamine in a carbonate buffer solution. Independently, a 12.5 mg/mL sSMCC solution in carbonate buffer was also prepared. Approximately 249 μL of the sMCC solution was mixed with 1 mL of the histamine solution and shaken for 1 hour at room temperature.

Preparation of the iminothiolane solution: A flask was charged with 0.25-1 mL of a 17.2 mg/mL 2-iminothiolane solution in carbonate buffer and then vortexed gently to dissolve the solids.

Preparation of thiolated sheep anti-tetracycline: To the tet solution prepared above, 23.13 μL of 2-Iminothiolane solution was added. The reaction vessel was sealed and gently shaken for 1 hour. Conjugation was achieved by slow addition of the activated histamine solution to the thiolated tet solution with mixing. The pH was adjust to 7.1 using a 0.5 M monobasic phosphate buffer solution. The reagents were then mixed overnight at 2-8° C. The volume of the resulting solution was approximately 1.773 mL. Excess reagents were removed by dialysis against a 50 mM phosphate buffer solution.

It should be apparent that the foregoing relates only to the preferred embodiments of the present invention and that numerous changes and modifications may be made herein without departing from the spirit and scope of the invention as defined by the following claims and equivalents thereof.

What is claimed is:

1. A device for measuring an amount of an analyte in a sample, comprising a lateral flow matrix which defines a flow path and which comprises in series:
a sample receiving zone;
a labeling zone comprising an unlabeled receptor and a labeled receptor, the unlabeled receptor located downstream of the labeled receptor and separated by a distance; and
two serially oriented capture zones capable of providing quantitation of the amount of the analyte in the sample.

2. The device of claim 1, wherein the unlabeled receptor comprises an antibody having a binding affinity for the analyte in the sample.

3. The device of claim 2, wherein the antibody is sheep anti-tetracycline.

4. The device of claim 1, wherein the unlabeled receptor comprises a cross-linked antibody having a binding affinity for the analyte in the sample.

5. The device of claim 4, wherein the cross-linked antibody comprises a monoclonal antibody species that is cross-linked to an antibody species.

6. The device of claim 5, wherein the monoclonal antibody species is different from the antibody species.

7. The device of claim 5, wherein the monoclonal antibody species is rabbit IgG and the antibody species is sheep anti-tetracycline.

8. The device of claim 4, wherein the cross-linked antibody comprises a small molecule tag that is cross-linked to an antibody species.

9. The device of claim 8, wherein the small molecule tag is histamine and the antibody species is sheep anti-tetracycline.

10. The device of claim 1, wherein the labeled receptor is bound to a detectable reagent.

11. The device of claim 1, wherein the labeled receptor is bound to detectable microparticles.

12. The device of claim 1, wherein the labeled receptor comprises a colloidal gold-conjugated antibody species.

13. The device of claim 12, wherein the colloidal gold-conjugated antibody species has gold particles in the range of about 20 nm to about 60 nm.

14. The device of claim 1, wherein the labeled receptor comprises an antibody species conjugated to latex particles.

15. The device of claim 14, wherein the antibody species conjugated to latex particles has latex particles in the range of about 20 nm to about 600 nm.

16. The device of claim 1, wherein the analyte is an antibiotic commonly found in foodstuffs.

17. The device of claim 16, wherein the antibiotic is selected from the group consisting of tetracyclines, beta lactams, quinolones, aminoglycosides, cephalosporins, macrolides, nitrofurans, and sulfonamides.

18. The device of claim 16, wherein the analyte is tetracycline.

19. The device of claim 1, wherein the analyte is a toxin commonly found in foodstuffs.

20. The device of claim 19, wherein the toxin is selected from the group consisting of mycotoxins, shellfish toxins, and pesticides.

21. The device of claim 1, wherein the distance is about 5 mm to about 50 mm.

22. The device of claim 1, wherein the lateral flow matrix further comprises a cellulosic membrane material.

23. The device of claim 1, wherein the device detects the analyte at a sensitivity in the range of about 5 ppb to about 1500 ppb.

24. The device of claim 1, wherein the device detects the analyte at a sensitivity in the range of about 10 ppb to about 150 ppb.

25. The device of claim 23, wherein an increase in the distance decreases the sensitivity.

26. The device of claim 1, wherein a result that the analyte is present in the sample at or above a threshold level is a positive result.

27. A method for measuring an amount of an analyte in a sample comprising:
providing a lateral flow matrix device comprising an unlabeled receptor and a labeled receptor, the unlabeled receptor located downstream of the labeled receptor and separated by a distance;
contacting the sample to the lateral flow matrix device, wherein the analyte binds to at least one of the unlabeled receptor or the labeled receptor to form one or more analyte-receptor complexes;
allowing the sample to come into contact with a receptor binder on a solid support, wherein the receptor binder binds to the at least one of the unlabeled or the labeled receptors but does not bind to the one or more analyte-receptor complexes; and
detecting a quantity of the receptor binder bound to the at least one of the unlabeled or labeled receptors as an inverse indication of the amount of the analyte in the sample at or above a predetermined threshold level.

28. The method of claim 27, wherein the unlabeled receptor comprises an antibody having a binding affinity for the analyte in the sample.

29. The method of claim 28, wherein the antibody is sheep anti-tetracycline.

30. The method of claim 27, wherein the unlabeled receptor comprises a cross-linked antibody having a binding affinity for the analyte in the sample.

31. The method of claim 30, wherein the cross-linked antibody comprises a monoclonal antibody species that is cross-linked to an antibody species.

32. The method of claim 31, wherein the monoclonal antibody species is different from the antibody species.

33. The method of claim 31, wherein the monoclonal antibody species is rabbit IgG and the antibody species is sheep anti-tetracycline.

34. The method of claim 30, wherein the cross-linked antibody comprises a small molecule tag that is cross-linked to an antibody species.

35. The method of claim 34, wherein the small molecule tag is histamine and the antibody species is sheep anti-tetracycline.

36. The method of claim 27, wherein the labeled receptor is bound to a detectable reagent.

37. The method of claim 27, wherein the labeled receptor is bound to detectable microparticles.

38. The method of claim 27, wherein the labeled receptor comprises a colloidal gold-conjugated antibody species.

39. The method of claim 38, wherein the colloidal gold-conjugated antibody species has gold particles in the range of about 20 nm to about 60 nm.

40. The method of claim 27, wherein the labeled receptor comprises an antibody species conjugated to latex particles.

41. The method of claim 40, wherein the antibody species conjugated to latex particles has latex particles in the range of about 20 nm to about 600 nm.

42. The method of claim 27, wherein the analyte is an antibiotic commonly found in foodstuffs

43. The method of claim 42, wherein the antibiotic is selected from the group consisting of tetracyclines, beta lactams, quinolones, aminoglycosides, cephalosporins, macrolides, nitrofurans, and sulfonamides.

44. The method of claim 27, wherein the analyte is tetracycline.

45. The method of claim 27, wherein the analyte is a toxin commonly found in foodstuffs.

46. The method of claim 45, wherein the toxin is selected from the group consisting of mycotoxins, shellfish toxins, and pesticides.

47. The method of claim 27, wherein the distance is about 5 mm to about 50 mm.

48. The method of claim 27, wherein the lateral flow matrix further comprises a cellulosic membrane material.

49. The method of claim 27, wherein the method detects the analyte at a sensitivity in the range of about 5 ppb to about 1500 ppb.

50. The method of claim 27, wherein the method detects the analyte at a sensitivity in the range of about 10 ppb to about 150 ppb.

51. The device of claim 49, wherein an increase in the distance decreases the sensitivity.

52. The method of claim 27, wherein a result that the analyte is present in the sample at or above a threshold level is a positive result.

53. The method of claim 27, wherein the lateral flow matrix device further comprises a control zone.

54. The method of claim 53, wherein the control zone comprises a control binder characterized in that it binds both to the at least one of the unlabeled or the labeled receptors and to the one or more analyte-receptor complexes.

55. The method of claim 54, further comprising detecting a quantity of the at least one of the unlabeled or the labeled receptors and to the one or more analyte-receptor complexes bound to the control binder.

56. The method of claim 53, wherein the lateral flow matrix device further comprises a test zone.

57. The method of claim 56, wherein the test zone comprises the quantity of the receptor binder bound to the at least one of the unlabeled or labeled receptors.

58. The method of claim 57, wherein the step of detecting further comprises comparing a first signal obtained from the test zone with a second signal obtained from the control zone, the method configured to provide a positive result when the analyte is present at or above the predetermined threshold level, wherein the positive result is indicated by a more intense second signal as compared to the first signal.

59. A kit for detecting the presence of a predetermined threshold amount of an analyte in a sample comprising:
a container comprising:
a lateral flow matrix which defines a flow path and which comprises in series:
a sample receiving zone;
a labeling zone comprising an unlabeled receptor and a labeled receptor, the unlabeled receptor located downstream of the labeled receptor and separated by a distance; and
two serially oriented capture zones capable of providing quantitation of the amount of the analyte in the sample,
wherein a result that the analyte is present in the sample at or above the predetermined threshold amount is a positive result; and
an incubator.
